(19) United States
(12) Patent Application Publication
(10) Pub. No.: US 2007/0036743 A1
(43) Pub. Date: Feb. 15, 2007

(54) Antimicrobial polymers

(75) Inventors: Krystyna Plochocka, Scotch Palins, NJ (US); Karen Winkowski, Springfield, NJ (US); Jeffrey Lynn, West Milford, NJ (US)

Correspondence Address: Attn: William J. Davis, Esq.;INTERNATIONAL SPECIALTY PRODUCTS, Legal Department, 1361 Alps Road, Building No. 8, Wayne, NJ, 07470, US

(73) Assignee: ISP INVESTMENTS INC.

(21) Appl. No.: 11/201,223

(22) Filed: Aug. 10, 2005

(51) Int. Cl. A61K 31/787; C08L 35/00

(52) U.S. Cl. 424/78.21; 525/207

(57) ABSTRACT

An antimicrobial polymer which is stable to aqueous hydrolysis contains a phenolic antimicrobial agent covalently bounded to one or more polymerized ethylenically unsaturated monomer units preferably through an imide and/or amide group.

### CROSS-REFERENCE TO RELATED U.S. PATENTS AND APPLICATIONS

This application is related to U.S. Pat. Nos. 5,869,695; 5,886,194; 5,959,122; 5,994,385; 6,025,501; U.S. Ser. No. 10/233,838, filed Aug. 30, 2002; Ser. No. 10/353,390, filed Jan. 29, 2003; and Ser. No. 10722,787, filed Nov. 26, 2003, all assigned to the same assignee as herein.

### BACKGROUND OF THE INVENTION

1. Field of the Invention

This invention relates to antimicrobial polymers, and compositions and delivery systems thereof, and, more particularly, to aqueous hydrolysis-resistant antimicrobial polymers which contain a phenolic group covalently bonded to the main chain of the polymer preferably through an imide and/or amide linkage.

2. Description of the Prior Art

Newington, I. et al, in U.S. Pat. No. 5,532,290, described antimicrobial polymers containing a phenolic antimicrobial agent bound through an anionic quaternary nitrogen atom to one or more polymerized ethylenically unsaturated monomer units. These ionic quaternary polymers were used as sanitizing agents in aqueous solutions, e.g. to clean surfaces such as bathroom and kitchen surfaces. However, such polymers are disadvantageous for certain applications because the ionic quaternary ammonium group is relatively unstable to aqueous hydrolysis over a long-term period, i.e. they do not provide adequate antimicrobial protection for building materials where very long-term resistance to moisture or rain is essential.

U.S. Pat. No. 2,875,097 disclosed antimicrobial agents used to make fabrics resistant to fungi and insects. U.S. Pat. No. 4,908,381 described polymers derived from ethylenically unsaturated monomers having side chains terminating in a pyran derivative which released glutaraldehyde upon contact with water.

Accordingly, it is an object of this invention to provide an antimicrobial polymer which will maintain its antimicrobial activity in the presence of water or rain over a long term period.

Another object of the invention is to provide stable antimicrobial polymers having a phenolic group covalently bonded to the main chain of the polymer preferably through an imide and/or amide linkage.

Another object of the invention is to provide an antimicrobial composition which is particularly suitable for use in protecting building materials from bacteria and/or fungi.

Still another object herein is to provide such antimicrobial compositions which can be used in delivery systems.

These and other objects and features of the invention will be made apparent from the following description.

### SUMMARY OF THE INVENTION

What is described herein is an antimicrobial polymer which is very stable to aqueous hydrolysis and which contains a phenolic antimicrobial agent covalently bounded to one or more polymerized ethylenically unsaturated monomer units preferably through an imide and/or amide linkage.

### DETAILED DESCRIPTION OF THE INVENTION

A. The antimicrobial polymers of the invention has the following general formula: 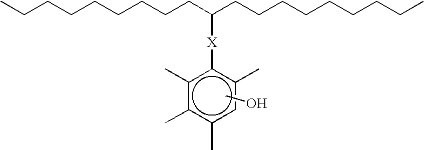
main polymer chain —X— is a single bond phenol-containing group or spacer group.

A preferred formula of the invention includes a phenol-group attached to the main polymer chain through an amide or imide bond, 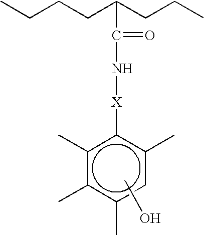
or maleimide bond.

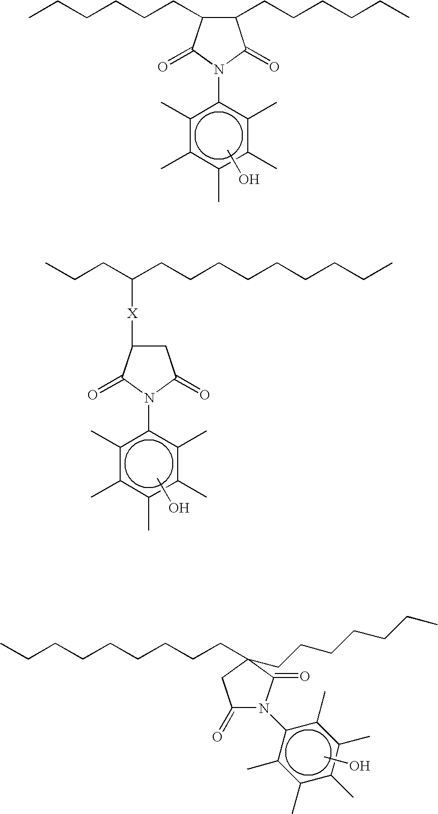

Other substituents which may be present in the main polymer chain include a carboxylic group, an ester group, particularly alpha-carboxyamide: 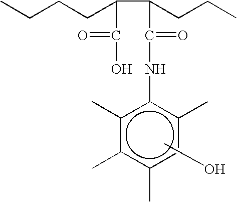
and alpha-carboxy ester

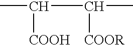

The main chain of the polymer can contain other optional monomer units, such as

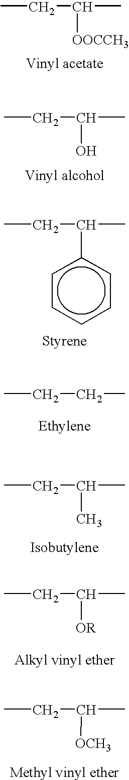

A preferred polymer of the invention has the general formula: 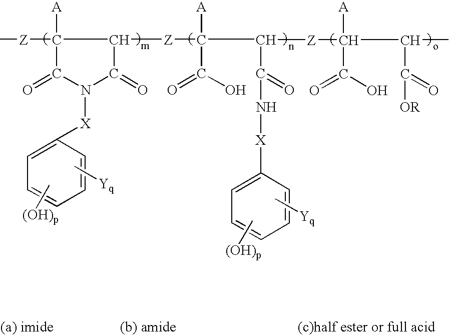
where m, n and o are present, in mole %, of 0-100; 0-50 and 0-99.5, with the proviso that at least one of m and n are present respectively;

R is H or alkyl;

A is H, alkyl, halogen or haloalkyl;

X is a single bond or a spacer group; e.g. alkylene, alkyleneoxy, silicone or alkylene carbonate;

Y is hydrogen, halogen, alkyl, thionyl, nitro or aromatic ring;

Z is an optional comonomer, alkene, vinylpyrrolidone, vinylcaprolactam, vinyl acetate, alkyl vinyl ether, styrene;

p is 1-5;

q is 1-3, and

p+q≦5.

A representative polymer has the formula: 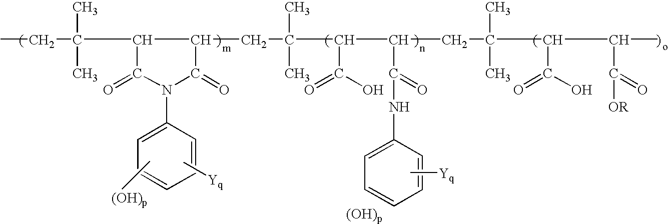
where m is 40-100; n is 0-50, and 0 is 0-50; Y is chloro or nitro and q is 0-3.

The antimicrobial polymers of the invention may be made by reacting an anhydride, acid or half-ester side group-containing polymer, e.g. Gantrez® AN (maleic anhydride), IBT, IB/MAN (isobutylene-maleic anhydride), VP/MAN (vinylpyrrolidone/maleic anhydride), vinyl acetate/maleic anhydride, itaconic acid or anhydride, with an aminophenol, e.g. an amino cresol, amino resorcinol, aminonapthol, and the like. In this process, the amine group of the phenol reacts with the anhydride to form a stable imide and/or amide linkage, while its —OH group remains free to kill fungi, mold, mildew and other microorganisms.

### Preparation of Polymers of Invention

1. Reaction with Copolymer of Maleic Anhydride 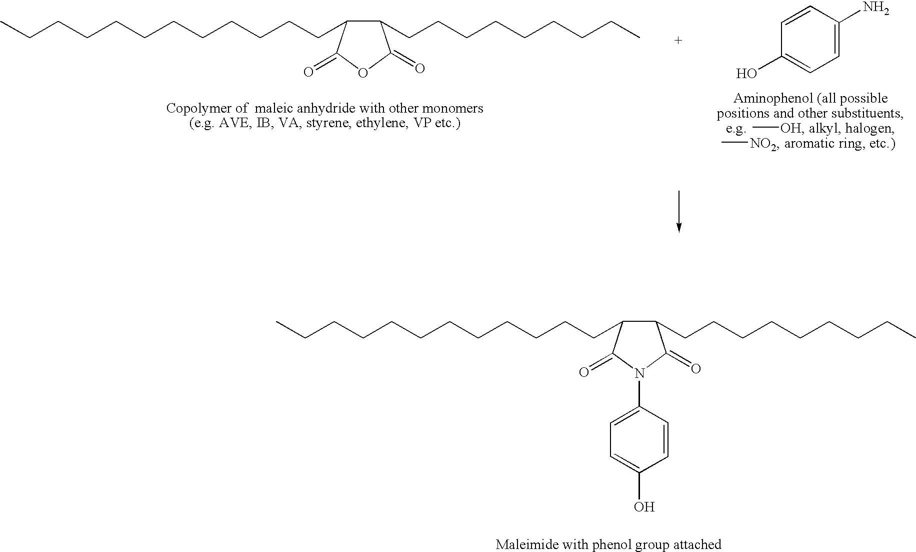
2. Reaction with Copolymer Containing Alpha Carboxy Ester Groups:

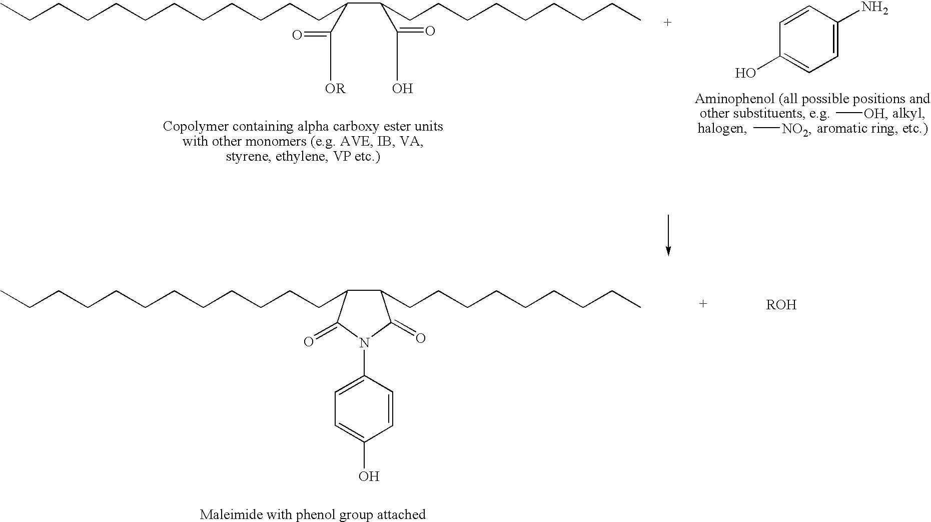

Antimicrobial polymers containing such aromatic (phenolic) —OH side groups of the invention are used effectively as antifouling materials for building materials and other solid surfaces, coated thereon or incorporated therein.

The antimicrobial polymers herein are even more active if the phenolic—OH is bound to the main chain via a spacer group. Suitable spacers include alkylene, oxyalkylene (e.g. EO or PO), polypropyleneoxy, polyethyleneoxy, silicone, etc. A preferred spacer is a —CH₂— unit.

Suitable polymers can be prepared by reacting an aminophenol with itaconic anhydride monomer, to produce the desired itaconic imide and/or amide.

Similarly, alkyl itaconates can be used as starting materials for preparing the antimicrobial polymers of the invention.

Other polymers containing a 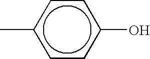
group can be made by reacting amino phenols, optionally substituted with a —SO₂Cl group, e.g. with a chloride of styrene sulfonic acid or a chloride of vinyl sulfonic acid. This reaction will generate preferentially a sulfonamide, which is stable to hydrolysis. Then polymerization follows.

Similar monomers can be made by reacting —SO₂Cl with phenol, or bis-phenol, where only one —OH group is reacted.

Other monomers in the copolymer can be selected to provide desired properties for the polymer.

The antimicrobial polymers or copolymers of the invention are particularly advantageous in commercial use because they are resistant to hydrolysis whereupon the phenol —OH group present therein can manifest its antimicrobial or antifouling activity over a prolonged period without regard to the effect of rain or other forms of moisture. Furthermore, the phenol group is permanently covalently bonded to the main chain of the biocidal polymer through a stable imide and/or amide bond. Thus the invention polymer will prevent microbial colonization in or on a substrate material, e.g. a building material, such as a shingle or gypsum board, while also killing fungi on its surface, or plastics, e.g. polyvinyl chloride materials, and can be delivered alone or with a drug in a delivery system.

The invention will now be described by reference to the following examples, in which:

### EXAMPLE 1

### Antibacterial Polymer Containing p-Aminophenol

77 g IB/MAN (1:1) copolymer (isobutylene/maleic anhydride) (0.5 mol equivalent of anhydride unit) and 250 g ethanol were charged into 1-liter Parr reactor. The reactor was sparged with nitrogen. The reaction was heated to 100° C. over 1 hour and the temperature was held for 4 hours; the ethyl half-ester was generated in this step. Then the reactor was cooled down to room temperature and left overnight. Thereafter 43.6 g (0.4 mole) of 4-amino-phenol dissolved in 65 g ethanol was added. The reactor was again sparged with nitrogen and heated to 100° C. over 0.5 hour and held at that temperature for 3 hours. Then the temperature was increased to 130° C. and held there for 8 hours. The reactor then was cooled to room temperature and discharged. The product was a brownish solution containing 27.6 wt. % solids. Based on ¹³C NMR analysis, it contained 74 mole % imide and less than 1 mole % unreacted amino-phenol.

### EXAMPLE 2

### Antibacterial Polymer Containing 4-Amino-2,6-Dichlorophenol

77 g IB/MAN (1:1) copolymer (0.5 mol equivalent of anhydride unit) and 400 g ethanol were charged into 1-liter Parr reactor. The reactor was sparged with nitrogen. The reaction was heated to 100° C. over 1 hour and the temperature was held for 5 hours; the ethyl half-ester was generated in this step. Then the reactor was cooled to room temperature and left overnight. Then 53.4 g (0.3 mole) of 4-amino-2,6-dichlorophenol (powder) was added. The reactor was again sparged with nitrogen and heated to 100° C. over 0.5 hour and held for 3 hours. Then the temperature was increased to 130° C. and held for 8 hours. Thereafter the reactor was cooled down to room temperature and discharged. The product was a brownish solution containing 25.2 wt. % solids. Based on ¹³C NMR analysis, it contained 49 mole % imide and 1 mole % unreacted amino-phenol.

### EXAMPLE 3

### Antibacterial Polymer Containing 2-Amino-6-Chloro-4-Nitrophenol

115.5 g IB/MAN (1:1) copolymer (0.75 mol equivalent of anhydride unit) and 345 g ethanol were charged into 1-liter Parr reactor. The reactor was sparged with nitrogen. The reaction was heated to 100° C. over 1 hour and the temperature was held for 6 hours; ethyl half-ester was generated in this step. Then the reactor was cooled down to room temperature and left overnight. Then 47.0 g (0.25 mole) of 2-amino-2,6-dichloro-4 nitrophenol (powder). The reactor was again sparged with nitrogen and heated to 100° C. over 0.5 hour and held for 6 hours. Then the reaction temperature was raised to 110° C. and held for 6 hours, then to 130° C. and held for 6 hours. Thereafter the reactor was cooled to room temperature and discharged. The product was a brownish solution containing 25.2 wt. % solids. Based on ¹³C NMR analysis, it contained 10 mole % imide and 40 mole % unreacted amino-phenol. The sample was purified by ultrafiltration before bio-testing.

### Biological Activity of Invention Examples on Gypsum Boards

A. The derivitized polymers of the invention examples were diluted in dimethyl sulfoxide (DMSO) to contain 100 or 1000 ppm of total solids. Then the grey side of a gypsum board sample (2×2×½ in.) was brush coated with each test formulation and allowed to dry for 24 hours. The control was a gypsum board treated with DMSO alone. The thus-treated gypsum boards were placed onto a Petri dish and water was added to saturate the sample. The water-saturated gypsum samples were then inoculated with a mixed fungal inoculum (Aspergillus niger, Penicillium funiculosum and Stachybotrys chartarum) containing e.g. 10⁵ spores/ml. The samples then were incubated at 28° C./80% RH for 30-45 days and rated for the presence (+) or absence (−) of fungal growth on the surface. The results are shown in Table 1 below.

TABLE 1
Fungal Growth Data

| Test Sample | Amt (ppm) | Activity |
|---|---|---|
| Control (DMSO) | | + |
| Example 1 | 100 | + |
| Example 1 | 1,000 | − |
| Example 2 | 100 | − |
| Example 2 | 1,000 | − |

B. The derivatized polymer of Example 3 was used to coat paper disks. The disks were allowed to dry for 24 hours and placed on the surface of Tryptic Soy Agar (TSA). 10 ml of the TSA containing the following microorganisms to a total concentration of about 10⁶ cells/ml was pipetted onto the surface of the plates containing the disks treated disks: Pseudomonas aeruginosa (ATCC 10145) or a mixed fungal inoculum containing Penicillium funiculosum (ATCC 11797) and Aspergillus niger (ATCC 6275) the plates containing the bacterial species were incubated at 32° C. for 48 hours. The plates containing fungal species were incubated at 28° C., 85% RH for 5-7 days. At the end of the incubation period the extent of growth on the surface of the disk was rated as + (growth) or − (no growth). The results are shown in Table 2 below.

TABLE 2

| Active Ingredient | P. aeruginosa | Mixed Fungal |
|---|---|---|
| Control (Untreated) | + | + |
| Isobutylene/maleic | + | + |
| anhydride copolymer | | |
| Example 3 | − | − |

While the invention has been described with particular reference to certain embodiments thereof, it will be understood that changes and modifications may be made which are within the skill of the art.

What is claimed is:

1. An antimicrobial polymer stable to aqueous hydrolysis containing a phenolic antimicrobial agent covalently bounded to one or more polymerized ethylenically unsaturated monomer units through an amide and/or imide group.

2. An antimicrobial polymer according to claim 1 wherein said ethylenically unsaturated monomer unit is maleic anhydride, an alkyl half-ester and/or full acid thereof.

3. An antimicrobial polymer according to claim 1 wherein said ethylenically unsaturated monomer unit comprises itaconic anhydride, its alkyl half-ester and/or its full acid.

4. An antimicrobial polymer according to claim 1 wherein said ethylenically unsaturated monomer unit comprises a copolymer of at least two monomer units.

5. An antimicrobial polymer according to claim 1 wherein said polymer is covalently bonded through an imide group.

6. An antimicrobial polymer according to claim 1 wherein said ethylenically unsaturated monomer unit is selected from maleic anhydride or itaconic anhydride, optionally including a comonomer selected from an α-olefin, vinyl acetate, alkyl vinyl ether, α-unsaturated carboxylic acid (meth)acrylic acid or its ester, vinyl pyrrolidone, vinyl caprolactam and styrene.

7. An antimicrobial polymer according to claim 1 wherein said phenolic antimicrobial agent is an aminophenol, an aminocresol, an amino resorcinol or an aminonapthol.

8. An antimicrobial polymer according to claim 1 wherein said phenol is bound to the main chain of the polymer via a spacer group.

9. An antimicrobial polymer according to claim 9 wherein said spacer group is an alkylene, oxyalkylene, or silicone.

10. An antimicrobial polymer according to claim 1 which has the formula: 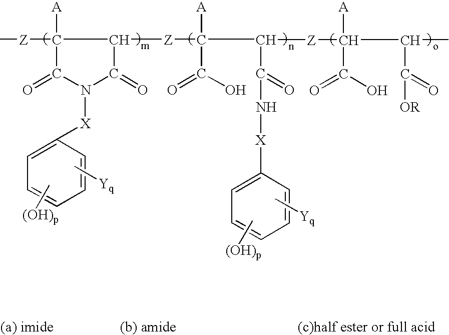
where m, n and o are present, in mole %, of 0-100; 0-50 and 0-99.5, with the proviso that at least one of m and n are present respectively;
R is H or alkyl;
A is H, alkyl, halogen or haloalkyl;
X is a single bond or a spacer group; e.g. alkylene, alkyleneoxy, silicone or alkylene carbonate;
Y is hydrogen, halogen, alkyl, thionyl, nitro or aromatic ring;
Z is an optional comonomer, alkene, vinylpyrrolidone, vinylcaprolactam, vinyl acetate, alkyl vinyl ether, styrene;
p is 1-5;
q is 1-3, and
p+q≦5.

11. An antimicrobial polymer according to claim 10 which has the formula: 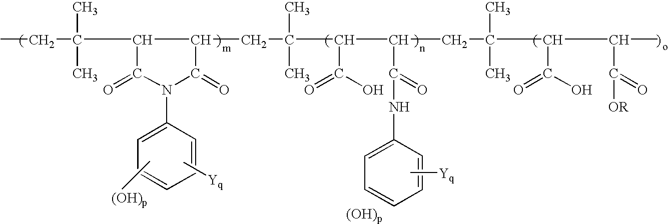
where m is 40-100; n is 0-50, and 0 is 0-50.

12. An antimicrobial polymer according to claim 10 which has the formula:

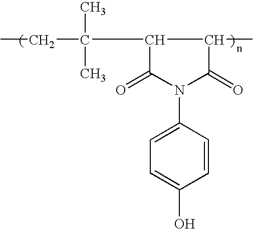

13. An antimicrobial polymer according to claim 10 wherein Y is a halogen and/or a nitro group.

14. An antimicrobial polymer according to claim 10 which is the p-aminophenol derivative of isobutylene-maleic anhydride.

15. An antimicrobial polymer according to claim 10 which is the 4-amino-2,6-dichlorophenol derivative of isobutylene-maleic anhydride copolymer.

16. An antimicrobial polymer according to claim 10 which is the 2-amino-2,6-chloro-4-nitrophenol derivative of isobutylene-maleic anhydride copolymer.

17. A composition including the antibacterial polymer of claim 1.

18. A building material product containing the antibacterial polymer of claim 1.
